(19) United States
(12) Patent Application Publication
(10) Pub. No.: US 2012/0005765 A1
(43) Pub. Date: Jan. 5, 2012

(54) ANIMAL MODEL FOR PARKINSON'S DISEASE

(75) Inventors: Vijaya Kumar, St. Louis, MO (US); William Burke, St. Louis, MO (US); Michael Panneton, St. Louis, MO (US)

(73) Assignee: Saint Louis University, Saint Louis, MO (US)

(21) Appl. No.: 13/175,617

(22) Filed: Jul. 1, 2011

Related U.S. Application Data
(60) Provisional application No. 61/360,911, filed on Jul. 1, 2010.

(51) Int. Cl. A01K 67/033; G01N 33/566; G01N 33/53; C12N 15/113; A61K 49/00

(52) U.S. Cl. 800/3; 536/24.5; 800/9; 435/7.1; 436/501

(57) ABSTRACT

Disclosed are methods and compositions for an animal model of Parkinson's disease. In particular, disclosed is the use of antisense compounds to inhibit the expression of ALDH1A1 in the substantia nigra of an animal brain for the purpose of creating an animal that will displays the symptoms of a human with Parkinson's Disease, including various biochemical, histological, and behavioral characteristics. Also disclosed are methods for using the animal model for Parkinson's disease to test potential therapeutic agents for Parkinson's disease.

### CROSS REFERENCE TO RELATED APPLICATION

This application claims priority to provisional application 61/360,911, filed Jul. 1, 2010, hereby incorporated by reference in its entirety.

### FIELD OF THE INVENTION

The invention relates to methods and compositions, for modifying gene expression or enzyme function to create an animal model for Parkinson's disease, and methods of using the animal model for the screening of therapeutic agents.

### BACKGROUND

Parkinson's disease (PD) is the second most common neurodegenerative disease (Bennett et al., (1996) New Engl. J. Med. 334, 71-76). The loss of dopamine containing neurons in the substantia nigra has been implicated in causing some symptoms of Parkinson's disease, including rigidity, bradykinesia, and tremor. However the mechanisms underlying this neuronal loss in Parkinson's disease are poorly understood. One reason for this is the lack of an appropriate, physiologically relevant animal model for Parkinson's disease. This lack of a relevant PD model makes it difficult to test for drugs which may be neuroprotective and inhibit the progression of PD. Most prominent Parkinson's disease animal models rely on the use of agents such as rotenone, 1-methyl-4-phenyl-1,2,3,6-tetrahydropyridine (MPTP), and 6-hydroxydopamine; all these substances are exogenous compounds not normally found in the organism. There is a long felt need for a physiological relevant animal model without exogenous agents to study non-genetic idiopathic Parkinson's disease.

It has been shown that 3,4-dihydroxyphenylacetaldehyde (DOPAL) is increased in PD brains compared to controls. In addition, aldehyde dehydrogenase (ALDH), the metabolic enzyme which converts DOPAL to a nontoxic product, 3,4-dihydroxyphenylacetic acid (DOPAC) in the substantia nigra is decreased in these PD brains (Mattammal M B et al. J (1993) Chromatog 614:205-212). Thus agents which decrease ALDH1A1, the specific ALDH isozyme found in the substantia nigra neurons (SN), should provide a physiologically relevant model of PD with which to test drugs for PD. The metabolism of dopamine by monoamine oxidase (MAO) has been implicated in neuronal loss of dopamine cells in the substantia nigra. The oxidation of dopamine produces a potential source of free radicals, including 3,4-dihydroxyphenylacetaldehyde (DOPAL) (Li et al., (2001) Mol Brain Res 93: 1-7,). There are several mechanisms by which DOPAL levels may be increased in dopamine neurons in the substantia nigra in Parkinson's disease. First, levels of mRNA, protein, and activity of aldehyde dehydrogenase (ALDH1A1), the enzyme which converts DOPAL to a nontoxic metabolite 3,4-dihydroxyphenylacetic acid (DOPAC), is decreased in SN neurons in Parkinson's disease (Marchitti et al., (2007) Pharmac Rev 59:125-150; Gaiter et al., (2003) Neurobiol of Disease 14:637-647). Second, mitochondrial complex I, the source of NAD, a cofactor for ALDH, is decreased in the SN in PD (Schapira et al., J Neurochem 54:823-827, 1990). For instance, pesticides like rotenone, which inhibit complex I and have been linked to PD (Betarbet et al., (2000) Nature Neurosci 3:1302-1306), also increase DOPAL levels and kill cells in vitro (Lamensdorf et al., (2000) Brain Res 868:191-201; Lamensdorf et. al., (2000) J. Neurosci. 60, 552-558).

The Inventors have previously disclosed the chemical synthesis of DOPAL (Li et al., (1998) Bio Org. Chem. 26:45-50). Here they disclose methods and compositions related to a physiologically relevant animal model for Parkinson's disease which presents decreased ALDH1A1 activity and increased DOPAL levels consistent with symptoms observed in Parkinson's disease patients. Also presented are methods of using the Parkinson's disease model for screening of therapeutic agents useful in treating Parkinson's disease.

### SUMMARY OF THE INVENTION

Disclosed are methods and compositions related to oligonucleotides directed at inhibiting ALDH1A1 expression in mammals.

Also disclosed are animal models for Parkinson's disease with reduced ALDH1A1.

Also disclosed are methods of making animal models for Parkinson's disease with reduced ALDH1A1.

Also disclosed are methods of using animal models for Parkinson's disease with reduced ALDH1A1 for testing potential therapeutic agents for the treatment of Parkinson's disease.

### REFERENCE TO COLOR FIGURES

The application file contains at least one figure executed in color. Copies of this patent application publication with color photographs will be provided by the Office upon request and payment of the necessary fee.

### DESCRIPTION OF THE FIGURES

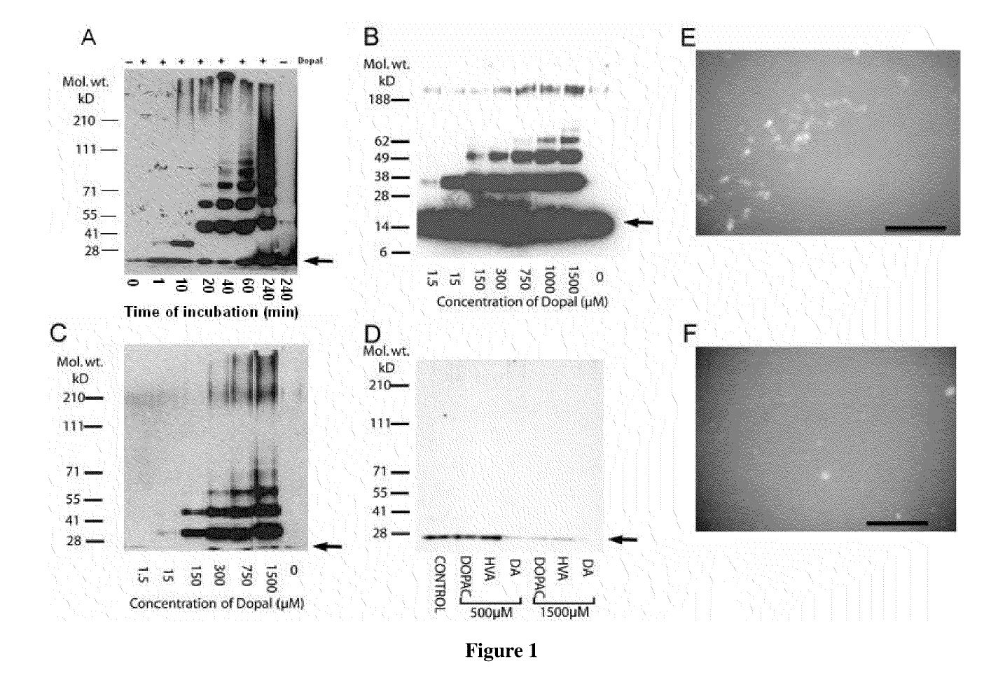

FIG. 1 shows Western blot detection of DOPAL induced aggregation of α-synuclein using a mouse monoclonal antibody to α-synuclein in a cell free system. One μg of α-synuclein (2 μM) was incubated with or without 1.5 mM DOPAL for various times and was electrophoresed on a tris-acetate gel (A). α-Synuclein (2 μM) was incubated for 60 min with or without increasing concentrations of DOPAL was and electrophoresed on bis-tris (B) or tris-acetate gels (C). One μg of α-Synuclein (2 μM) was incubated for 60 min with or without increasing concentrations of DA, or its metabolites DOPAC and HVA, and electrophoresed on tris-acetate gel (D). After PAGE, immunoblotting was performed as described in Methods. (Arrows in A-D show monomer with MW of 17 kD.). Fluorescence microscopy of thioflavin-S stained, DOPAL induced α-synuclein aggregates is shown in E and F. α-Synuclein (2 μM) was incubated with (E) or without (F) 1.5 mM DOPAL for 4 h. The incubation mixtures were stained with thioflavin-S and viewed under a fluorescence microscope. Scale bar 100 mM.

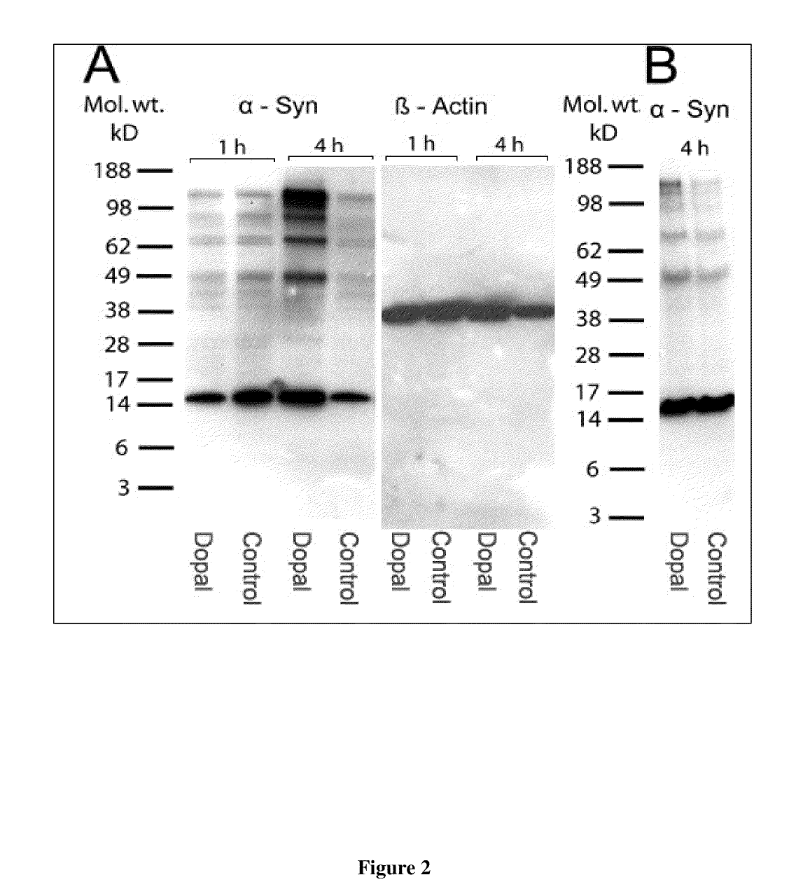

FIG. 2 shows Western blot of extracts from rat SN injected with DOPAL. (A) SN was injected with either 1.0 μg DOPAL or vehicle control (lanes 2, 4). After either 1 hr or 4 hrs, rats were sacrificed, the SN biopsied and, immunoblotted using AS 202 monoclonal antibody. The blot was stripped and reprobed with β-actin antibody. (B) SN was injected with either 0.2 μg DOPAL or vehicle control, and after a 4 hr, immunoblotting was performed using AS 202 antibody as described above. Compare the DOPAL dose effect after 4 hrs in (A) to that in (B). It was noted that α-synuclein aggregation is dependent on the DOPAL dose but even small amounts of DOPAL still aggregate α-synuclein.

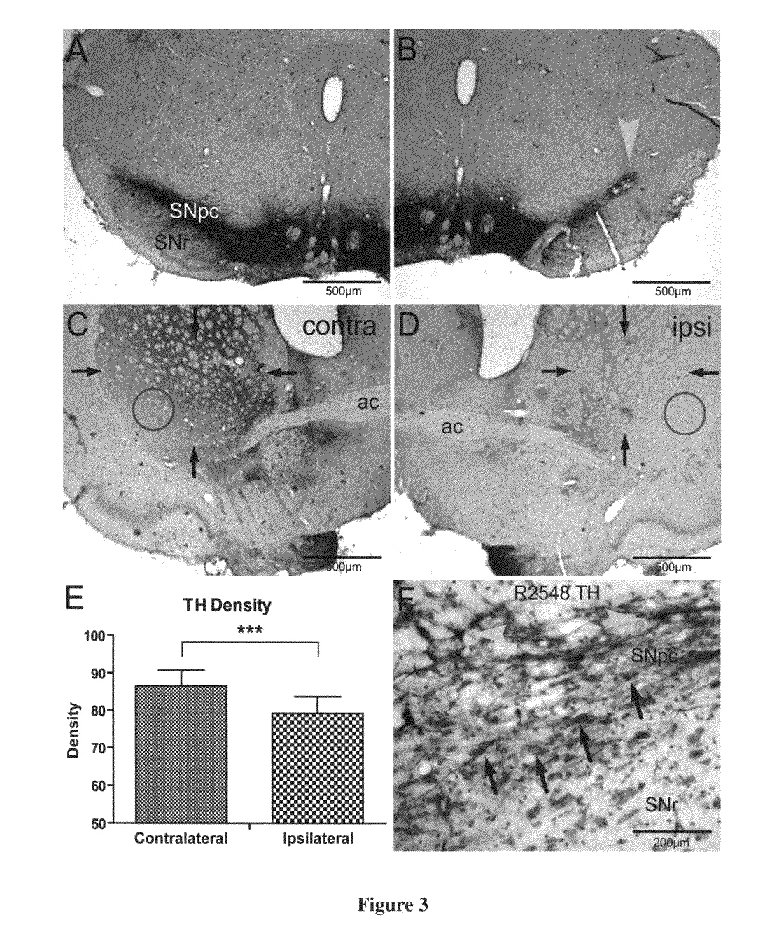

FIG. 3 shows photomicrographs of brain sections from a rat immunohistochemically-stained against tyrosine hydroxylase (TH) after injections of DOPAL into the substantia nigra, pars compacta (SNpc). Note the gross reduction of TH immunoreactivity in the SN at the site of injection (B; yellow arrowhead) versus the non-injected side (A). Similar loss of TH staining is seen in the striatum ipsilateral to the injection (D, arrows) versus that on the non-injected side (C, arrows), suggesting disruption of nigral dopaminergic terminals. The area just lateral to the anterior commissure (D, yellow arrowhead) however was always densely labeled (see text). Densitometry of immunostaining of striatal TH (E) showed significant differences (p, 0.001) of the whole striatum contralateral and ipsilateral to DOPAL injections. Spot density measurements of ventrolateral parts of the striatum (D, red circles), however, showed an 80% loss of immunoreactivity ipsilateral to the injection. Intensely stained neurons with antibodies against tyrosine hydroxylase (F, yellow arrowheads) were sometimes seen in the SNpc of control brains surrounded by numerous neurons stained only for Nissl (F, black arrows), suggesting that counting only TH-immunostained neurons may be problematic. Abbreviations: ac, anterior commissure; SNpc, pars compacta of substantia nigra; SNpr, pars reticulata of substantia nigra. *** p, 0.001.

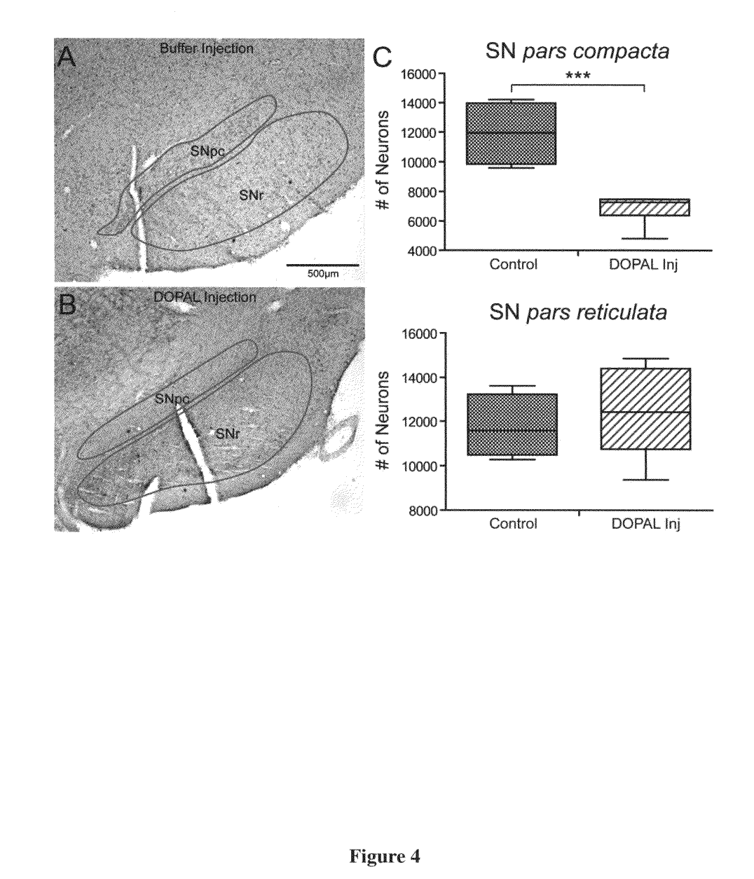

FIG. 4 shows photomicrographs of sections through the SN stained for Nissl with neutral red. Red lines mark the boundaries enclosing the substantia nigra, pars compacta, while green lines encompass the substantia nigra, pars reticulata. Unbiased stereological counts using optical fractionator probes were made of neurons in both SNpc and SNpr in sections from animals injected either with buffer (A) or with DOPAL (B). Note the significant (p=0.001) loss of SNpc neurons in rats after the DOPAL injection when compared to control rats, while no loss of neurons was seen in the adjacent SNpr (C).

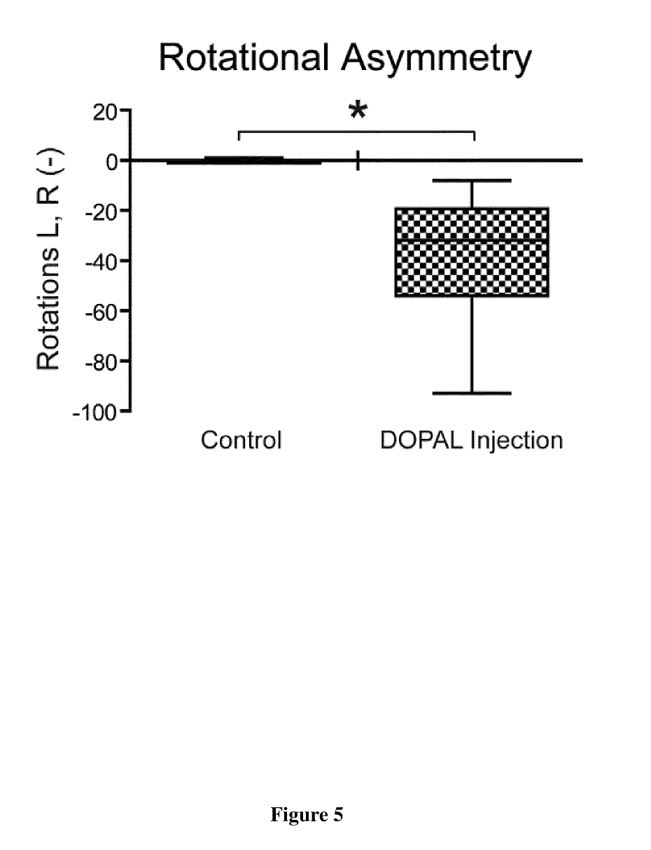

FIG. 5 shows a box plot illustrating the behavioral changes in rats after unilateral injections of DOPAL into their substantia nigra. Rats showed rotational asymmetry, turning significantly towards the side of DOPAL injections. *p, 0.05.

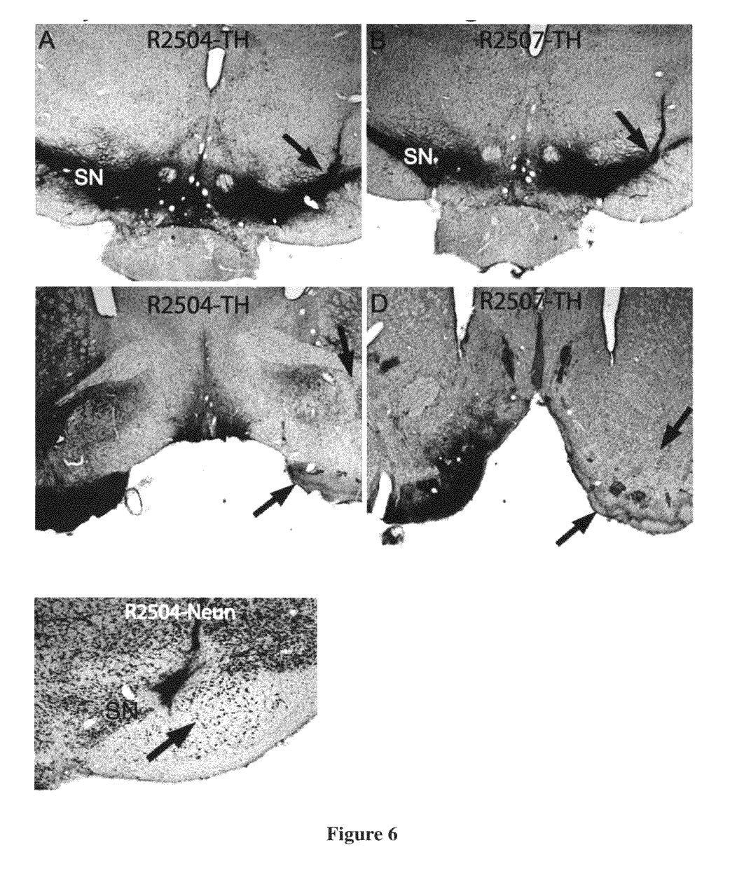

FIG. 6. shows photomicrographs of sections of rat brains stained immunohistochemically after injections of antisense oligonucleotide against ALDH1A1 on DA SN neurons. Three unilateral injections (total 800 nl; 700 pg/nl) of an antisense compound (SEQ ID NO: 1) against the enzyme ALDH1A1 were made into the substantia nigra nucleus of the rat. Sections from two different rats are stained immunohistochemically for tyrosine hydroxylase (TH) and (A-D). The injections (arrows) shown in A (R2504; sacrificed 5 weeks after injection) and B (R2507; sacrificed 7 weeks after injection) were placed in the pars compacta portion of the substantia nigra, the subnucleus where most of the dopaminergic neurons are found. It was noted that the dopamine projections to the ventral striatum are absent/decreased on the side ipsilateral to the injections of antisense nucleotide (C, D; arrows) when compared to the opposite non-injected side, suggesting loss of dopamine. An adjacent section showing neuronal cell bodies immunostained with antibodies against Neun is shown from case R2504. It was noted the loss of immunostaining in the area immediately adjacent to the injection site (marked with red beads), suggesting that neurons here have died, but numerous neurons in the subjacent pars reticulata of the SN (arrow) remain.

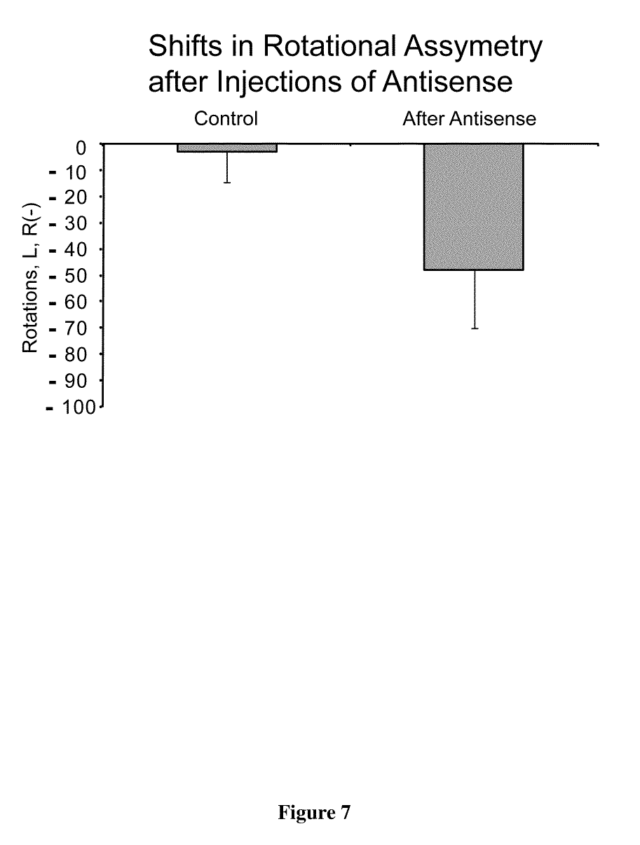

FIG. 7. shows a bar graph illustrating the behavioral changes in rats after unilateral injections of antisense oligonucleotides against ALDH1A1 mRNA into their substantia nigra. Rats (n=7) showed rotational asymmetry, turning significantly towards the side of antisense injections. This behavior mimicked that seen after DOPAL injections and implies a loss of striatal dopamine.

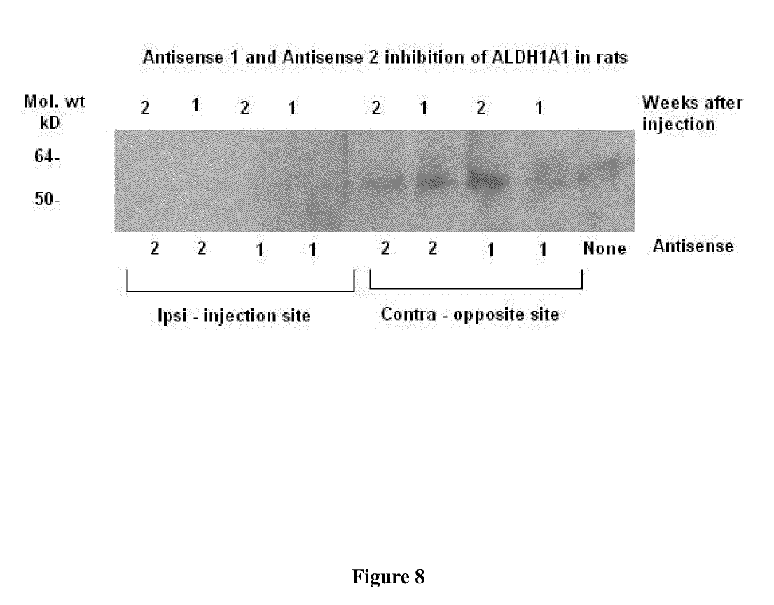

FIG. 8 shows Western blot of rat substantia nigra from rats injected with ALDH1A1 antisense 1 (SEQ ID NO: 1 and antisense 2 (SEQ ID NO: 3) Rats were injected weekly with two different oligonucleotides against ALDH1A1 mRNA and tissue harvested after 1-2 weeks. Apparent was the loss of ALDH1A1 protein on the side ipsilateral to the injections compared to that on the contralateral non-injected side, suggesting the antisense molecules disrupted synthesis of ALDH1A1 protein. . . .

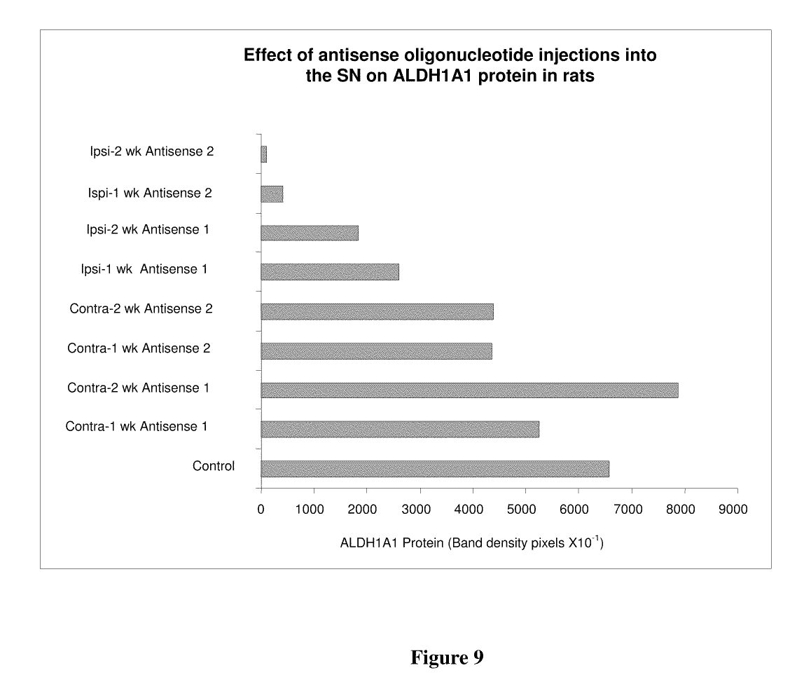

FIG. 9 shows the quantification of the Western blot results obtained in FIG. 8 using optical density scanning technology. Bands obtained from the immunoblot of FIG. 8 were quantified by Unscan software (Silk Scientific) and the band densities plotted. Apparent was the loss of density on the side ipsilateral to the injections of both antisense oligonucleotides, but especially the antisense 2 molecule. This suggests the antisense molecules disrupted synthesis of ALDH1A1 protein in the substantia nigra.

### DETAILED DESCRIPTION OF THE INVENTION

Disclosed is a physiologically relevant animal model for Parkinson's disease achieved through the use of RNA interference to reduce expression of cytosolic aldehyde dehydrogenase 1A1 (ALDH1A1). While the etiology of Parkinson Disease (PD) is unknown, the Inventors have shown that increased levels of 3,4-dihydroxyphenylacetaldehyde (DOPAL) in the brain are related to Parkinson's disease (PD) symptoms and behaviors (Panneton et. al., (2010), PLOS One, 5, 12, e15251). While not wishing to be bound by theory the inventors hypothesize that the endogenous breakdown product of dopamine (DA) metabolism, DOPAL, acts as a toxin to kill dopaminergic neurons in the substantia nigra (SN), and leads to the pathology of PD (Burke et al., (1999) Anal. Biochem. 273, 111-116; Mattammal et al., (1995) Neurodegeneration 4, 271-281; Goldstein et al., (2011) European J. Neurol. 18, 703-710). The normal breakdown of DOPAL into non-toxic products by ALDH is impaired in PD individuals. Id.

Dopamine is metabolized by monoamine oxidase (MAO) into an aldehyde, DOPAL. DOPAL is further converted to its non-toxic metabolite, DOPAC in the SN by ALDH1A1 or by mitochondrial ALDH. Consequently, decreased ALDH1A1 activity should induce DOPAL to increase in the SN. The inhibition of ALDH in PC-12 cells in vitro induces DOPAL to accumulate intracellularly (Lamensdorf et al. (2000) Brain Res. 868, 191-201) and promotes cell loss. Moreover, ALDH1A1 is the isoform of ALDH that is specific to DA SN neurons and patients with sporadic PD show decreased ALDH1A1 in the SN (Mandel et al. (2007) Ann. NY Acad. Sci. 1053, 356-375.; Marchitti, et al., (2007) Pharmacol. Rev. 59, 125-150). Complex I, when inhibited by the pesticide rotenone which produces nicotine adenine dinucleotide, a cofactor for ALDH1A1, also is decreased in PD individuals (Schapira, (1990) J. Neurochem. 54, 23-27).

The inventors have demonstrated that DOPAL is highly toxic to DA neurons in vitro (Burke, et al. (2008) Acta Neuropathol. 115, 193-203) and in vivo (Panneton et. al., (2010), PLOS One, 5, 12, e15251) and those animals with elevated levels of DOPAL present many characteristic symptoms or disease indicators for PD. By way of example, increased DOPAL triggers the formation of hydroxyl radicals in the presence of hydrogen peroxide that activate mitochondrial permeability transition pores leading to cell death (Li et al., (2001) Mol Brain Res 93:1-7; Kristal et al., (2001) Free Rad Biol & Med 30: 924-933; Burke et al., (2003) Brain Res. 989, 205-213). Free radicals also trigger aggregation of α-synuclein (AS) (Hashimoto, et al. (1999) Neuroreport 10, 717-721). DOPAL, also triggers formation of AS oligomers and large Lewy body-like aggregates (Burke, et al., (2008) Acta Neurpoathol. 115, 193-203; Galvin et al., (2006) Acta Neuropath. 112, 115-126). Also, AS oligomers further induce pore-forming proteins to release DA from storage vesicles (Voiles et al., (2002) Biochemistry. 41, 4595-602; Voiles et al. (2001) Biochem. 40, 7812-7819), leading to increased DOPAL in the cytosol, and cell death (Kristal BS et al., (2001) Free Rad Biol & Med 30: 924-933, Moreover, DOPAL injections into the SN of rats induce PD-like behaviors in rodents, including rotational asymmetry (Panneton et. al., (2010), PLOS One, 5, 12, e15251). In summary, a causative agent in Parkinson's disease may be a decreased activity of ALDH1A1 leading to increased DOPAL levels and the death of the DA SN neurons.

A preferred Parkinson's disease animal model (PD animal model) reproduces the symptoms and disease indicators of PD as accurately as possible in an easily managed animal. Any non-human animal may be used as disease model for PD. Rodents are a preferred animal model for PD and the rat is most preferred since it has been well studied and it is easily trained and observed. Since it was believed that that PD patients have increased levels of DOPAL caused by reduced ALDH1A1 activity in the SN, it is desirable to create a rat model for PD with reduced levels of ALDH1A1 expression or ALDH1A1 activity. The Inventors injected antisense oligonucleotides into the SN of rats to decrease ALDH1A1 mRNA and protein expression. These injections decreased/eliminated ALDH1A1 protein (FIG. 8), increased PD-like behavior similar to DOPAL (FIG. 7) and reduced dopaminergic neurons in the SN (FIG. 6).

### I. RNA Interference

Regulation of mRNA to control the expression of a specific protein may be performed by different molecular techniques including RNA interference. One RNA interference technique that functions in a variety of systems is antisense oligonucleotide technology. (Wagner, (1994) Nature, 372:333-335; Wagner et al., (1995) Science, 26:1510-1513; Smith et al., (1988) Nature, 334:724-726; Symons, (1989) Trends in Biochem. Sci., 14:445-452; and Kumar et al. (2000) Peptides 12:1769-1775). However, merely knowing complementary sequences for a particular mRNA does not provide a method for selecting antisense oligonucleotides with the desired regulatory effects at a useful potency. (See, e.g., Wagner, (1994) Id. at 334). Moreover, the physiological effects of the administration of antisense oligonucleotides to cells and/or animals cannot be predicted a priori.

### A. Sequences

A number of RNA interference devices may be employed to prevent or inhibit the production or translation of the specific mRNA message for ALDH1A1 provided they share complementation with ALDH1A1 mRNA or its precursors. A Parkinson's disease animal model may be made from any convenient non-human animal comprising target sequences that hybridize with ALDH1A1 antisense oligonucleotides and result in a reduction of ALDH1A1 mRNA or protein. A preferred animal is the rat. The nucleotide sequence for rat ALDH1A1 mRNA is represented by its corresponding deoxyribonucleic acid (cDNA) sequence, NM_{—}022407.3 (SEQ ID NO: 2). Rat ALDH1A1 mRNA also may be represented by conservatively modified variants of SEQ ID NO: 2. The mRNA sequence for rat ALDH1A1 may be derived from the corresponding cDNA sequence set forth in NM_{—}022407.3 (SEQ ID NO: 2) by substituting uracil (U) for thymine (T).

An antisense oligonucleotide is complementary to the chosen target nucleic acid so as to specifically hybridize to the target. Designing an antisense oligonucleotide compound begins with identifying target nucleic acids whose function is to be modulated. Target nucleic acids of the disclosed PD model include ALDH1A1 mRNA and its precursors. An antisense oligonucleotide sequence may be complementary to any particular coding or non-coding region of ALDH1A1 mRNA or to ALDH1A1 pre-mRNA. During expression, the ALDH1A1 gene is transcribed to pre-mRNA which undergoes post-transcriptional splicing to produce ALDH1A1 mRNA which is translated into protein. Antisense oligonucleotides may interfere with any step in ALDH1A1 expression including: transcription of the ALDH1A1 gene to ALDH1A1 pre-mRNA; post-transcriptional splicing of ALDH1A1 pre-mRNA to ALDH1A1 mRNA; or translation of ALDH1A1 mRNA into protein. An antisense oligonucleotide may bind to pre-mRNA or mRNA thereby blocking expression, and/or signaling its degradation by ribonucleases. In a rat model of PD, an antisense oligonucleotide complementary to a coding or non-coding region of a nucleotide encoding rat ALDH1A1 pre-mRNA may interfere with post-transcriptional splicing and production of ALDH1A1 mRNA. In a preferred rat model of PD, antisense oligonucleotides that are complementary to coding or non-coding regions of a nucleotide encoding rat ALDH1A1 mRNA (SEQ ID NO: 2) block translation of ALDH1A1 mRNA into protein, and/or signal degradation of ALDH1A1 mRNA by ribonucleases.

It is not necessary for an antisense oligonucleotide to be 100 percent complementary to the target nucleic acid to be effective. It is expected that an antisense oligonucleotide that is complementary to an entire or less than an entire target sequence of a given nucleic acid may be effective in reducing the levels of ALDH1A1 mRNA and consequently ALDH1A1 protein. Antisense oligonucleotides effective in reducing levels of ALDH1A1 are complementary to at least 8; more preferably at least 10; more preferably at least 12; more preferably to at least 14; even more preferably to at least 18; yet most preferably to at least 22 nucleotides of one or more of the target nucleic acids. Antisense oligonucleotides effective in reducing levels of ALDH1A1 may also be complementary to more than 22 and as long as 42 nucleotides of the target nucleic acids. For examples see Kumar et al. (2000) Peptides 12:1769-1775 and U.S. Pat. No. 6,310,048, incorporated herein by reference in their entirety.

It is preferable that antisense oligonucleotides be complementary to a specific site or sites within the target nucleic acid sequence for the oligonucleotide interaction to occur and have the desired effect, namely, reduced expression of ALDH1A1. An antisense oligonucleotide may be complementary to coding or non-coding target nucleic acids. Coding nucleic acids code for protein whereas non-coding nucleic acids may provide for functional or regulatory roles. Antisense oligonucleotide may be complementary to nucleic acids that encode for polypeptide structure, and/or may be complementary to non-coding nucleic acids which may provide functional roles in ALDH1A1 expression, including transcription, post-transcriptional splicing, or translation. By way of example, target sequences of a nucleic acid related to polypeptide expression may include sequences at or near a ribosomal binding site, a protein start site, an internal or protein coding site and/or a protein stop site.

A preferred antisense oligonucleotide is complementary to a target nucleic acid sequence encoding the ALDH1A1 protein start sequence and/or adjacent sequences which are nearby, upstream, downstream or both, and reduces the expression of ALDH1A1. By way of example are antisense oligonucleotides that are complementary to target nucleic acids encoding the ALDH1A1 protein start sequence, including about 7 nucleotides upstream and about 12 nucleotides downstream of the “ATG” start codon, and in one such example, are complementary to the ATG start codon and surround nucleic acids residues positioned at residues 29 to 31 of SEQ IS NO: 2 (NM_{—}022407.3).

Preferred antisense oligonucleotides specifically hybridize to target nucleic acids of at least 8; more preferably at least 10; more preferably at least 12; more preferably to at least 14; even more preferably to at least 18; yet more preferably to at least 22; nucleic acids encoding the ALDH1A1 protein start sequence and/or sequences up stream or down stream. Antisense oligonucleotides may also hybridize to target nucleic acids encoding more then 22 target nucleic acids. It is also preferred that the antisense compound hybridize to nucleic acids that are contiguous. More preferred antisense oligonucleotides which share complementation with ALDH1A1 mRNA include any 8, 9, 10, 11, 12, 13, 14, 15, 16, 17, 18, 19, 20, 21, or 22 contiguous nucleotide-bases set forth in the sequence 5″-TGCAGGGGAAGACATTGCTGGT-3″ (SEQ ID NO: 1), and conservatively modified variants thereof. A most preferred example of an antisense oligonucleotide which shares complementation with the ALDH1A1 protein start region is a 22 contiguous deoxyribonucleotide-bases set forth as 5″-TGCAGGGGAAGACATTGCTGGT-3″ (SEQ ID NO: 1), preferred due to its effectiveness as an inhibitor of ALDH1A1 synthesis.

Another preferred antisense oligonucleotide is complementary to target nucleic acid sequences encoding ALDH1A1 protein and/or adjacent sequences which are nearby, upstream, downstream or both, and reduces the expression of ALDH1A1. By way of example, are antisense oligonucleotides that are complementary to target nucleic acids that encode any region of the ALDH1A1 protein, including a region that extends from about 285 nucleotides to about 305 nucleotides downstream of the “ATG” start codon, and in one such example, are complementary to the sequence set forth at residues 313 to 333 of SEQ IS NO: 2 (NM_{—}022407.3).

Preferred antisense oligonucleotides specifically hybridize to target nucleic acids of at least 8; more preferably at least 10; more preferably at least 12; more preferably to at least 14; even more preferably to at least 18; yet more preferably to at least 21; nucleic acids encoding ALDH1A1 protein and/or sequences up stream or down stream. Antisense oligonucleotides may also hybridize to target nucleic acids encoding more then 21 target nucleic acids. It is also preferred that the antisense compound hybridize to nucleic acids that are contiguous. More preferred examples of antisense oligonucleotides that are complementary to a sequence of nucleic acids encoding ALDH1A1 protein include any 8, 9, 10, 11, 12, 13, 14, 15, 16, 17, 18, 19, 20, or 21 contiguous nucleotide-bases set forth in the sequence 5″-AGCAGACGATCTCTCTCCATT-3″ (SEQ ID NO: 3), and conservatively modified variants thereof. A most preferred example of an antisense oligonucleotide which shares complementation with target nucleic acids encoding ALDH1A1 protein is a 21 contiguous deoxyribonucleotide-bases set forth as 5″-AGCAGACGATCTCTCTCCATT-3″ (SEQ ID NO: 3), preferred due to its effectiveness as an inhibitor of ALDH1A1 synthesis.

It is expected that oligonucleotide sequences that share partial identity with the sequences disclosed herein may also be successful in inhibiting ALDH1A1 expression. For example, sequences which share 99 percent, 98 percent, 97 percent, 96 percent, 95 percent, 90 percent, 85 percent, 80 percent or 70 percent identity with the sequences disclosed herein may be effective for inhibiting ALDH1A1 expression. Sequence identity or percent identity is intended to mean the percentage of same residues between two sequences.

To calculate percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It will be appreciated that a single sequence can align differently with other sequences and hence, can have different percent sequence identity values over each aligned region. It is noted that the percent identity value is usually rounded to the nearest integer. For example, 78.1%, 78.2%, 78.3%, and 78.4% are rounded down to 78%, while 78.5%, 78.6%, 78.7%, 78.8%, and 78.9% are rounded up to 79%. It is also noted that the length of the aligned region is always an integer.

The alignment of two or more sequences to determine percent sequence identity is performed using the algorithm described by Altschul et al. (1997, Nucleic Acids Res., 25:3389 402) as incorporated into BLAST (basic local alignment search tool) programs, and available at ncbi.nlm.nih.gov on the World Wide Web. BLAST searches can be performed to determine percent sequence identity between a nucleic acid molecule of the invention and any other sequence or portion thereof aligned using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a sequence of the invention and another sequence, the default parameters of the respective programs are used. Sequence analysis of nucleic acid sequences can be performed used BLAST version 2.2.9 (updated on May 12, 2004).B. Antisense compounds and modified oligonucleotide backbones

While antisense oligonucleotides comprised of DNA, or RNA are a preferred form of antisense compound, the present invention contemplates other oligomeric antisense compounds, including, but not limited to, oligonucleotide mimetics those containing modified backbones (which may be referred to herein as “modified internucleoside linkages”). As defined herein, oligonucleotides having modified backbones include those that retain a phosphorous atom in the backbone, as well as those that do not have a phosphorous atom in the backbone. Modified oligonucleotide backbones which are useful in the subject antisense oligonucleotides include, for example, phosphorothioates, chiral phosphorothioates, phosphotriesters, aminoalkylkphosphotriesters, methyl and other alkyl phosphonates including 3′-alkylene phosphonates and chiral phosphonates, phosphinates, phosphoramidates including 3′-aminophosphoramidate and aminoalkylphosphoramidates, thionophosphoramidates, thionoalkylphosphonates, and boranophosphonates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. A preferred antisense compound backbone is a phosphorothioate.

Various salts, mixed salts and free acid forms are also included. References that teach the preparation of such modified backbone oligonucleotides are provided, for example, in U.S. Pat. No. 5,945,290. Modified oligonucleotide backbones that do not include a phosphorous atom therein may comprise short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. References that teach the preparation of the oligonucleotides listed above are provided in U.S. Pat. No. 5,945,290.

Other useful oligonucleotide mimetics, which are useful in the subject antisense oligonucleotides, comprise replacement of both the sugar and the internucleoside linkage—i.e., the backbone-of the nucleotide units with novel groups. One such oligomeric compound that has excellent hybridization properties is a peptide nucleic acid. See, e.g., Nielsen et al., Science, 254:1497-1500 (1991); and U.S. Pat. Nos. 5,539,082; 5,714,331; and 5,719,262. In such peptide nucleic acid compounds the sugar-backbone of an oligonucleotide is replaced with an amide containing backbone, in particular with an aminoethylglycine backbone. The nucleobases are retained and are bound directly or indirectly to aza nitrogen atoms of the amide portion of the backbone. Other useful modified oligonucleotides are those having phosphorothioate backbones and oligonucleotides with heteroatom backbones, and in particular —CH₂—NH—O—CH₂—, —CH₂—N(CH₃)—O—CH₂—, —CH₂—O—N (CH₃)—CH₂—, —CH₂—N(CH₃)—N(CH₃)—CH₂—, and —O—N(CH₃)—CH₂—CH₂—, wherein the native phosphodiester backbone is represented as —O—P—O—CH₂—, (as disclosed in U.S. Pat. No. 5,489,677), and the amide backbones disclosed in U.S. Pat. No. 5,602,240. Also useful are oligonucleotides having morpholino backbone structures as taught in U.S. Pat. No. 5,304,506.

Modified oligonucleotides can also contain one or more substituted sugar moieties (which may be referred to herein as “modified sugar moieties”). Useful oligonucleotides comprise one of the following at the 2′ position: OH; F; O—, S—, N-alkyl; N-alkenyl; N-alkynyl; or O-alkyl-O-alkyl, wherein the alkyl, alkenyl, or alkynyl may be substituted or unsubstituted C1 to C10 alkyl, or C2 to C20 alkenyl and alkynyl; O(CH₂)O(CH₃); O(CH₂)O(CH₂)ₙCH₃; O(CH₂)nNH₂; or O(CH₂)ₙCH₃ (where n=l to 10); Cl; Br; CNB; CF₃; OCF₃; NO₂; N₃; NH₂, heterocycloalkyl; heterocycloalkaryl; aminoalkylamino; polyalkylamino; substituted silyl; an RNA cleaving group; a cholesterol group; a reporter group; an intercalator; a group for improving the pharmacokinetic properties of an oligonucleotide; or a group for improving other substituents having similar properties. Oligonucleotides can also have sugar mimetics such as cyclobutyls in place of the pentafuranosyl group. A preferred modified sugar moiety is a 2′-O-methoxyethyl sugar moiety.

Other useful antisense compounds may include at least one nucleobase modification or substitution. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases, such as 5-methylcytosine, 5-hydroxymethylcytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocystine, 5-halouracil and cytosine, 5-propynyl uracil and cytosine, 6-azo uracil, cytosine and thymine, 5-uracil, 4-thiouracil, 8-halo, 8-amino, 8-thiol, 8-thioalkyl, 8-hydroxyl and other 8-substituted adenines and guanines, 5-halo, particularly 5-bromo, 5-trifluromethyl and other 5-substitutes uracils and cytosines, 7-methylguanine and 7-methyladenine, 8-azaguanine and 8-azaadenine, 7-deazaguanine and 7-deazaadenine and 3-deazaguanine and 3-deazaadenine.

The antisense compounds of the present invention may be conveniently and routinely made through the well-known technique of solid phase synthesis. Equipment for such synthesis is available from several manufacturers and vendors including, for example, Applied Biosystems, Foster City, Calif. Any other means for such synthesis known in the art may additionally or alternatively be employed. It is also well known to use similar techniques to prepare modified oligonucleotides such as the phosphorothionates and alkylated derivatives that are discussed above.

### C. Formulations

A “pharmaceutically acceptable carrier” (also referred to herein as an “excipient”) is a pharmaceutically acceptable solvent, suspending agent or any other pharmacologically inert vehicle for delivering one or more of the subject antisense oligonucleotides to an vertebrate. The pharmaceutically acceptable carrier may be a liquid or a solid and is selected with the planned manner of administration in mind so as to provide for the desired bulk, consistency, and other pertinent transport and chemical properties, when combined with one or more of the subject antisense oligonucleotides and any other components of a given pharmaceutical composition. Typical pharmaceutically acceptable carriers include, but are not limited to, saline solution; binding agents (e.g., pregelatinized corn starch, polyvinylpyrrolidone or hydroxypropyl methylcellulose, or etc.); fillers (e.g., lactose and other sugars, microcrystalline cellulose, pectin, gelatin, calcium sulfate, ethyl cellulose, polyacrylates orcalcium hydrogen phosphate, and the like); lubricants (e.g., magnesium stearate, starch, polyethylene glycol, sodium benzoate, sodium acetate, and the like); disintegrates (e.g., starch, sodium starch glycolate, and the like); or wetting agents (e.g., sodium lauryl sulfate, and the like).

The pharmaceutical compositions of this invention may be administered in a number of ways depending upon whether local or systemic treatment is desired, and upon the area to be treated. Administration may be topical (including opthalmic, rectal, intranasal, transdermal), oral or parenteral, for example, by intravenous drip, subcutaneous, intraperitoneal or intramuscular injection or intrathecal or intraventricular administration, such as, for example, by intracerebral ventricular injection (ICV) or bilateral or unilateral injection into the substantia nigra. It is believed that the subject antisense oligonucleotides also can be administered by tablet, since the toxicity of the oligonucleotides is very low. Administration can be either rapid as by injection or over a period of time as by slow infusion or administration of slow release formulations. For treating tissues in the central nervous system, administration can be by injection or infusion into the cerebrospinal fluid.

Antisense oligonucleotide can be coupled to any substance known in the art to promote uptake by a target cell or tissue such as by way of non-limiting example an antibody to the transferrin receptor, and administered by intravenous injection.

The subject antisense compounds may be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. For example, cationic lipids may be included in the formulation to facilitate oligonucleotide uptake. One such composition shown to facilitate uptake is LIPOFECTIN (available from GIBCOBRL, Bethesda, Md.).

The antisense compounds of the present invention can include pharmaceutically acceptable salts, esters, or salts of such esters, or any other compound which, upon administration to an animal, including a human, is capable of providing—directly or indirectly—the biologically active metabolite or residue thereof. Accordingly, for example, the invention is also meant to include prodrugs and pharmaceutically acceptable salts of the compounds of the invention, pharmaceutically acceptable salts of such prodrugs, and other bioequivalents.

As used herein, the term “prodrug” means a therapeutic agent that is prepared in an inactive form that is converted to an active form within the body or cells thereof by the action of endogenous enzymes or other chemicals and/or conditions.

An oligonucleotide may be administered which are processed to provide antisense oligonucleotides for the purposes of reducing levels of ALDH1A1 or protein. By way of example, an oligonucleotide may be administered that is designed to be transcribed produce an antisense oligonucleotide capable of hybridization with the target nucleic acid.

The term “pharmaceutically acceptable salts” means physiologically and pharmaceutically acceptable salts of the compounds of the invention: i.e., salts that retain the desired biological activity of the parent compound and do not impart undesired toxicological effects thereto.

The present invention also includes pharmaceutical compositions and formulations that include the antisense compounds of the invention. Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets or tablets. Thickeners, flavorings, diluents, emulsifiers, dispensing aids or binders may be desirable. Formulations for parenteral administration may include sterile aqueous solutions which may also contain buffers, diluents and other suitable additives.

In addition to the use of antisense, it is also anticipated that other methods which reduce ALDH1A1 activity may be used to create a PD animal model. For example, any of sequences disclosed herein may be adapted to the various forms of RNA interference including but not limited to small RNAs, small interference RNAs and microRNAs and methods of delivery which may used reduce the expression of ALDH1A1. In addition, animals may be genetically engineered so that they lack expression of, or exhibit reduced expression of ALDH1A1, by deleting or interfering with the sequences disclose herein. These genetically engineered animals are commonly referred to as transgenetic or knock out animals, and have been described by Guerts et al., (2009 Science 325:433), and also in U.S. Pat. Nos. 7,038,105, 6,365,796, 7,309,811 and 6,552,246, all of which are hereby incorporated by reference in their entirety. It is also anticipated that the use of chemical agents which inhibit ALDH or ALDH1A1 may be used to create a PD animal model, for example daidzein, which has been shown to be effective at inhibiting ALDH in PC-12 cells by Lamensdorf I et al., (2000, Brain Res 868:191-201), hereby incorporated by reference in their entirety.

### II. Parkinson's Disease Animal Model

### A. Method of Making a Parkinson's Disease Animal Model

Any non-human animal may be administered an effective amount of antisense oligonucleotide compound to provide a Parkinson's disease animal model (PD animal). A preferred animal is a rodent; the most preferred rodent is a rat. The antisense oligonucleotide compound may be administered in any pharmaceutically acceptable carrier and any route of administration including those disclosed in section I.C. Preferably the antisense oligonucleotide is administered by injection into the SN. A most preferred method is injection unilaterally into the SN. A PD animal may be designed to test a particular putative or potential treatment for PD. A putative or potential treatment may include the administering an active substance or potential therapeutic agent. Once a potential therapeutic agent is selected, the dosage of the antisense oligonucleotide administered to produce the PD animal may be selected to model a more severe or less severe PD disease state, appropriate for the effectiveness of the putative or potential treatment. Effective amounts of antisense oligonucleotide will vary with the route of administration, and the severity of PD disease state desired. By way of example, effective amounts of antisense oligonucleotide include: from about 0.001 pg to about 0.01 pg, from about 0.01 pg to about 0.1 pg, from about 0.1 pg to about 1.0 pg, from about 0.001 ng to about 0.01 ng, from about from about 0.01 ng to about 0.1 ng, from about 0.001 μg to about 0.01 μg, from about 0.01 μg to about 0.1 μg, from about 0.1 μg to about 1.0 μg, from about 14 to about 10 μg, from about 10 μg to about 100 μg, from about 100 μg to about 1000 μg, from about 1 mg to about 10 mg, and from about 10 mg to about 100 mg per kilogram of non-human animal. Preferable, effective amounts are from about 0.01 μg to about 10 μg per kilogram, more preferable is about 0.1 μg to about 10 μg/kg, and most preferably about 1 μg to about 10 μg per kilogram of non-human animal. After 5-7 weeks, the PD disease state is established and the animals may be used for study or testing of potential therapeutic agents.

### B. Testing Potential Therapeutic Agents

The present invention provides a method of using the PD animal model for testing putative or potential treatments for Parkinson's disease including the in vivo activity of an active substance for treating Parkinson's disease. Once a PD disease state is established in an animal the animals they may then be subjected to a putative or potential treatment for example administration of an active substance or potential therapeutic agent. The protocol and route of administration will vary according to the mechanism of action and the chemical nature of the active substance and may be determined by those skilled in the art. An active substance can be combined with a pharmaceutical acceptable carrier and administered through any appropriate route of administration including those listed in section I. C.

PD animals subjected to putative or potential treatment for PD may be assessed by any appropriate method including behavioral, biochemical, and histological, for any possible effects of the putative or potential treatment and compared to PD animals that were not treated.

Examples of behavioral assessments are described in Ungerstedt and Arbuthnott (1970) 24, 3, 18 485-493; Konitsiotis et al. (1998), 92, 1, 77-83;Truong et al., (2006) 169, 1, 1-9), all of which are incorporated herein by reference. In one example rats are introduced to the behavioral test one week prior to treatment; 3 days after treatment and at selected intervals thereafter until the rats are sacrificed. In one example rats are timed walking over a balance beam 36 mm wide and 105 cm long elevated 80 cm from the floor. Their motivation is to join other rats in a cage at the end of the beam. Time for initiation of walking and as well as travel time is recorded with a hand-held stopwatch. Animals refusing to complete a test run are ‘timed out’ at 2 min. In another example, stepping patterns of rats are measured as they walk along a walkway (65 mm wide) attached to the balance beam. Their hind paws are dipped in black ink and their steps recorded on paper strips taped to the walkway. The distance between three consecutive left and right steps are recorded. This analysis of gait is monitored weekly. Another example includes testing for rotational asymmetry Rats are injected subcutaneously with the dopamine agonist apomorphine (0.4 mg/kg) dissolved in 0.1% ascorbate saline solution. After 5 minutes, they are placed in a hemispheric rotation bowl 40 cm wide and 20 cm deep and the number of complete turns to the right or the left quantified by observation. This test is performed both prior to injection and again at selected intervals thereafter up to sacrifice. Rotational asymmetry has become the standard for testing unilateral depletions of striatal dopamine in rodents, including from disruptions of nigrostriatal circuitry.

Behavioral assessments are useful in the screening of potential therapeutic agents for the treatment of Parkinson's disease. By way of general example, PD animals are treated with a potential therapeutic agent and after waiting an appropriate time period for the potential therapeutic agent to take effect, the PD-animal is injected with an effective amount of apomorphine, by way of example, 0.4 mg/kg apomorphine (subcutaneously), and rotational asymmetry assessed. By way of example, rotational asymmetry is observed for 30 min and the number of ipsilateral and contralateral turns quantified by observation. The PD animals perform this test one week prior to receiving antisense oligonucleotides, prior to receiving putative or potential treatment, and again after receiving putative or potential treatment. Other improvements in mobility may be compared including an enhanced amount or speed of movement, and/or a delayed outset of mobility impairment.

Non-limiting examples of biochemical assessments include, determination of DOPAL levels by any biochemical assay known in the art. Assessments also include cell based assays, for example cell toxicity, aggregation of purified α-synuclein, or aggregation of α-synuclein in culture and in vivo as described in the examples.

Examples of histologically assessments include loss of DA SN neurons and/or their basal forebrain projections and aggregation of α-synuclein in basal forebrain projections. Compounds that may have utility in treating Parkinson's disease can be identified using this approach.

In one preferred embodiment are oligonucleotide sequences complementary to ALDH1A1 mRNA which when administered to a mammalian cell or an animal through any method of RNA interference including but not limited to antisense, small RNAs, small interference RNAs and microRNAs, will reduce the expression of ALDH1A1.

In one preferred embodiment is an antisense compound, complementary to ALDH1A1 mRNA that when administered to an animal reduces ALDH1A1 expression and produces an animal which exhibits a Parkinson's like disease state.

In another embodiment is a non-human animal, preferably a rat, having been administered an antisense compound, complementary to ALDH1A1 mRNA, exhibits reduced ALDH1A1 expression and a Parkinson's like disease state.

In another embodiment is a non-human animal, preferably a rat, with reduced ALDH1A1 expression or increased DOPAL levels and a Parkinson's like disease state.

In another embodiment is a genetically engineered non-human animal, preferably a rat, with reduced ALDH1A1 expression and a Parkinson's like disease state.

In yet another embodiment is a method of testing a potential therapeutic agent for the treatment of Parkinson's disease by administering the potential therapeutic agent to any of the Parkinson's disease animal models described herein, and comparing Parkinson's disease symptoms to control Parkinson's disease animal model animals not receiving the potential therapeutic agent

As used herein, the term “antisense compound” is meant to include, but not be limited to, antisense oligonucleotides, and is intended to include antisense oligonucleotides that are chemically modified or other chemical compounds that specifically bind to the same targeted nucleic acids that are described herein, and that provide the same regulatory effect on ALDH1A1 expression as the subject antisense oligonucleotides. The antisense oligonucleotides of the present invention are synthesized in vitro and do not include antisense oligonucleotides of biological origin, except for oligonucleotides that comprise the subject antisense oligonucleotides and which have been purified from or isolated from such biological material. The antisense compounds in accordance with this invention preferably comprise from about 8 to about 22 nucleotides or longer than 22 nucleotides. However, an antisense compound of even fewer than 8 nucleotides, for example, a fragment of the preferred antisense compound is understood to be included within the present invention so long as it demonstrates the desired activity of inhibiting the expression of ALDH1A1.

As used herein, the term “Parkinson's disease state” is meant to include any animal exhibiting symptoms of Parkinson's disease including but not limited to the behavioral, biochemical, and histological changes described herein.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLES

### Methods and Materials

Measurement of DOPAL, DA and HVA in human SN and striatum MHPLC separation of catecholamines with electrochemical detection. Microcolumn high performance liquid chromatography (MHPLC) was used in combination with a BAS LC-4C amperometric controller for electrochemical detection (EC) (BAS, West Lafayette, Ind.). The MHPLC system consisted of a Waters 515 HPLC pump (Waters, Milford, MA), Unijet amperometric detector cells and SepStik microbore reverse phase column (C 18, 5 urn, 150×1.0 mm id) with glassy carbon working electrode and AG/AgCl reference electrode. The whole system was controlled by a BAS D-5 controller and the results were recorded and calculated using a personal computer with ChromGraph software. The mobile phase for eluting all the metabolites of interest in the brain consisted of 0.073 M disodium phosphate, 0.027 M citric acid, 1.2 mM sodium heptanesulfonic acid, 0.2 mM EDT A, 9 mM sodium chloride and 3.5% acetonitrile. The pH of the solution was adjusted to 5.62. The solution was filtered through a 0.2 um filter and degassed before use. The separation was performed isocratically at a flow-rate of 40 μl/min with a sensitivity of 2 nA. The detector potential was set at 0.60 V vs Ag/AgCl. The injection volume was 3-10 μl. Total elution of all the analytes was completed within 30 minutes.

Extraction procedures for DOPAL and HVA from brain. Brain was obtained from a 67-year-old man with a post-mortem interval of 8.3 h. The brain was grossly and microscopically normal. Samples of brain (50 mg to 100 mg wet weight) from caudate, putamen and substantia nigra were placed in 1.5 ml polypropylene Eppendrof centrifuge tubes and 200 ul of 0.1 M perchloric acid (PCA) containing 50 ng of internal standard 3,4-dihydroxybenzylamine (DHBA) and 0.5% sodium metabisulfite as an antioxidant was added. The tissue was initially homogenized in the centrifuge tubes using a closely fitting 8 mm OD Teflon pestle. Tissue homogenates were maintained in an ice-bath for 10 min and centrifuged at 14,000 g in a microcentrifuge for 20 min. The supernatants were mixed with from 150 to 400 μl of 0.2M phosphate buffer (PH 8.97) as needed to bring pH to 7.5. The mixtures were applied to alumina N columns and allowed to flow slowly. The columns were washed with 3 to 4 ml of diethyl ether and eluted with 5 to 7 ml of ethyl acetate. The ethyl acetate extracts were concentrated to dryness under vacuum at 10° C. The residues were reconstituted in 200 ul 0.1 M PCA and filtered through a 0.2 urn Nylon-66 filter. Albumina B columns were used for extraction of DA from all tissues prior to injection. Aliquots of from 3 to 10 ul were injected into the MHPLC system.

Alumina extraction procedure for brain DA. An aliquot of brain tissue was homogenized in 200 μl of 0.1 M PCA containing 50 ng of internal standard and 0.5% sodium metabisulfite as an antioxidant. The mixture was centrifuged at 14,000 g for 20 min. The supernatant was added to 200 μl of Tris buffer (PH 8.6) and 20 mg of alumina (WB-5, Sigma) and mixed on a rotation shaker for 15 min. After centrifuging at 12,000 g for 5 min, the supernatant was discarded. The alumina was washed twice with 500 μl of distilled water. The catechols (CA) were eluted from the alumina twice by 150 μl of 0.1 M acetic acid. The combined eluates were evaporated under vacuum without heat. The residue was reconstituted in 100 μl 01 M PCA and filtered. Aliquots of 10 μl were injected onto the MHPLC system.

Calibration curves for quantitation of DOPAL, DA and HVA. Calibration curves were generated for DOPAL and all the compounds of interest vs DHBA as the internal standard. The concentration range used for the standard calibration curve was 10 pg/μl to 1000 pg/μl for all compounds tested with 250 pg/μl of internal standard (DRBA). A calibration curve was constructed by plotting the ratios of the peak height of DOPAL or other compounds tested to the peak height of DHBA. The relationship between the peak height and concentration was linear through the range tested. The linear regression line was determined by the least squares method. The average detectable limits were between 5 and 10 pg per injection.

Determination of DOPAL toxicity in vitro Toxicity experiments were essentially as described previously. PC12 cultures were initially expanded in T-75 flasks with 20 ml Dulbecco's Modified Eagle's Medium supplemented with 4.5 g/l glucose, 4 mM L-glutamine, 5% fetal calf serum, 10% horse serum, 1000 units/l penicillin, and 100 mg/l streptomycin. Cells were transferred to six well culture plates with 2 ml media and grown to 1.5×105 cells/well, treated with 50 ng/ml nerve growth factor (NGF), and maintained in NGF-containing media for 7 d prior to experimentation. The medium was changed every 2-3 d. NGF treatment caused the cells to differentiate, as judged by neurite outgrowth. During the experiments, the medium was replaced once a day. Cells were detached with buffered saline containing EDT A, centrifuged at 200×g and resuspended in trypan blue for counting of viable cells. To prevent extreme values from exerting undue influence on mean values, counts>4 standard deviations away from the mean of identically treated wells (3/59, ˜5%) were excluded from the data as described previously. DOPAL and other compounds were added as described in the description of the figure and examples. Data was analyzed by ANOVA.

Measurement of DOPAL toxicity in vivo. Injections and tissue preparation. The housing and nutrition of the rats and all procedures performed on them conformed to the standards set forth in the Guide for the Care and Use of Laboratory Animals of the National Research Council (National Academy Press, 1996). The experimental protocols reported here were reviewed and approved by the Animal Care Committee and monitored by the Department of Comparative Medicine of the Saint Louis University School of Medicine. The details of the surgical and immunohistochemical methods used have been published previously.

Briefly, male Sprague-Dawley rats (200-300 g; Harlan, Indianapolis, Ind. USA) were deeply anesthetized with 0.16 mill/100 g of a mixture of ketamine (100 mg/ml), zylazine (20 mg/ml) and saline (9:7:4, Lp.). The heads of the rats were fixed in a stereotaxic apparatus (David Kopf, Tujunga, CA, USA) and either DOPAL, DA, DOPAC, DOPET, HVA or the vehicle consisting of 1.0% benzyl alcohol in phosphate-buffered saline (PH 7.4) was injected unilaterally into the VTA and the substantia nigra compacta (SNC) of each by pressure through a 1.5 mm pipette pulled to a tip diameter of 30-50 μm. Rhodamine microspheres (1.0 μM; Molecular Probes, Eugene, Oreg., USA) were injected with the compounds to reveal the injection sites. Thirty-one rats were used and all of these received multiple injections involving the SN and VTA on one or both sides of the brain. The numbers of injections (n), involving the SN were as follows: DA-20 μg (2), 1 0 μg (2), 5 μg (2), 500 ng (4); DOPAL-500 ng (3) 250 ng (4), 100 ng (2), 50 ng (I); DOPAC-500 ng (2); DOPET-500 ng (2); HVA-500 ng (2); vehicle (I). The numbers of injections involving VTA were as follows: DA-20 μg (2), 10 μg (2), 5 μg (2), 500 ng (4); DOPAL-750 ng (1), 500 ng (2), 250 ng (3), 100 ng (3), 50 ng (2); DOPAC-500 ng (2); DOPET-500 ng (2); HVA-500 ng (2); Vehicle (1). Injections were made in volumes of 0.2 μl.

Immunohistochemistry. Eighteen hours (two rats) or 5 days following the surgery the rats were anesthetized and perfused through the left ventricle of the heart with 0.1 M phosphate buffer containing 4% paraformaldehyde. The brains were removed, post-fixed for at least 4 h, sunk in 25% sucrose, and sectioned frozen at 50 pm with a sliding microtome. Adjacent series of sections were subjected to a conventional immunoperoxidase protocol using antibodies against tyrosine hydroxylase (TH; Sigma, monoclonal, made in rat, used at a dilution of 1:6000), neuronal nuclear antigen (NeuN, Chemicon, Temecula, Calif., USA, made in rabbit, used at 1:20,000) or glial fibrillary acidic protein (GFAP, made in rabbit, used at 1:5000). Briefly, the sections were immersed overnight in 0.1 M Sorenson's phosphate buffer (SPB, pH 7.4) containing 0.2% Triton X-100 (SPB/Triton) and primary antibody with agitation. The following morning they were thrice rinsed in SPB/Triton and immersed for 1 h in SPB/Triton containing biotinylated secondary antibodies against the host species of the primary antibodies, used at a dilution of 1:200. The sections were again thrice rinsed in SPB/Triton and then immersed in SPB/Triton containing ABC reagents (Vector, Burlingame, Calif., USA) used at a dilution of 1:200. After further rinsing in SPB the sections were reacted with 3,3-diaminobenzidine (DAB) and hydrogen peroxide to produce an insoluble brown reaction product that was further intensified with osmium and thiocarbohydrazide as has been described. All immersions and rinses were done at room temperature. In addition, a series of sections from many of the brains was mounted and processed for Nissl staining using a standard cresyl violet staining procedure. Processing was concluded by cover slipping the sections under DPX (Fluka, Sigma-Aldrich, St. Louis, Mo., USA).

NeuN is a marker of neuronal differentiation that allows neurons to be distinguished immunohistochemically and thus demonstrates neuronal loss more clearly than Nissl-staining by avoiding the confounding issues of glia in the material. Antibodies against NeuN have been used as a convenient means to examine lesions following the injection of excitotoxins into the central nervous system.

Determination of DOPAL-triggered α-synuclein aggregation in vitro. Western blot of DOPAL-triggered AS aggregation in test tube experiments. DOPAL was dissolved in 1% benzyl alcohol, then diluted to a final concentration of 1.5-1,500 μM as described previously. AS (2 μM) was incubated at 37° C. in 20 μl of 100 mM tris-HCl buffer (pH 7.2) with or without DA, DOPAL, DOPAC, or homovanillic acid (HVA) for up to 4 h. The reaction was stopped by heating at 70° C. for 3 min in SDS buffer. The entire mixture was transferred to the appropriate gel (vide infra).

SDS-PAGE and immunoblotting. Ten micrograms of protein (estimated by Bio Rad protein reagent) was resolved by electrophoresis on 4-12% bis-tris gels run with MES SDS buffer, and 3-8% tris-acetate gels using tris-acetate running buffer. For immunoblotting, the protein was transferred to PVDF blotting membranes with 2 mm pore size, blocked with 5% milk protein in tris buffered saline (TBS) containing 0.1% Tween 20, and incubated for 1 h with a AS 202 monoclonal antibody (1:2,500 dilution). Blots were washed five times with 5% milk protein in TBS and probed with horseradish peroxidase-conjugated anti-mouse secondary antibody (1:2,500). Blots were washed five times with TBS containing. 0.5% Tween 20 before detection with Super Signal (Pierce).

Western blot of DOPAL-triggered AS aggregation in vivo Intranigral DOPAL injections and tissue preparation for Western blot. The housing and nutrition of the rats used in this study and all procedures performed on them conformed to standards set forth in the Guide for the Care and Use of Laboratory Animals of the National Research Council (National Academy Press, 1996). The experimental protocols reported herein were reviewed and approved by the Animal Care Committee of the Saint Louis University.

The details of the surgical and immunohistochemical methods have been previously described (Burke et al., Brain Res 989:205-213, 2003). Briefly, 2-month-old Sprague-Dawley rats (300 g; Harlan, Indianapolis, Ind., USA) were deeply anesthetized with 4% induction and maintained with 1% isoflurane. The heads of the rats were fixed in a stereotaxic apparatus (David Kopf, Tujunga, CA, USA). Single injections of DOPAL were made into the SN unilaterally (AP-5.5 mm, ML 2 mm and DV −7.4 mm from bregma) via a glass micropipette (O.D. 30-5-μm) glued to a 1-μl Hamilton syringe. Control injections of similar volumes of the vehicle (1.0% benzyl alcohol in phosphate-buffered saline, pH 7.4) were made on the opposite side. Three rats were injected with 400 nl/1.0 μg DOPAL, while six were injected with 200 nl/0.2 μg DOPAL. Black microspheres (6.0 μm; Molecular Probes, Eugene, Oreg., USA) were added to mark the injection sites.

Rats for Western blot analysis were killed after 4 h by injecting pentobarbital (100 mg/kg, IP). Their brains were exposed, the midbrain removed stereotaxically, and a punch of the injection site obtained using a IS-gauge needle. The resultant biopsies were frozen immediately on dry ice and stored at −80° C. until analysis. Tissue biopsies were homogenized in five volumes and centrifuged at 13,000 g for 10 min at 4° C. The black microspheres were identified in the pellets and supernatants were analyzed with Western blot as described above using AS 202 and J3-actin antibodies. For the 4-h 1.0-μg DOPAL and control SN injections, the density of the bands on the blot was determined using the UnScan it program (Silk Scientific, Orem, Utah, USA). The results were expressed as units of AS aggregate/unit of actin. See Section 4a for Western blot protocol.

Determination of DOPAL-induced Parkinson's disease-like behavior Injections of DOPAL or ALDH1A1 antisense oligo into rat SN. To determine if increased levels of DOPAL in SN produces a behavioral model of PD, 3 groups of rats were tested: unilateral SN injection of 4 μg DOPAL in 6 rats; unilateral SN injection of 0.56 μg antisense oligonucleotide (oligo) targeted to aldehyde dehydrogenase (ALDH1A1) mRNA, the DOPAL catabolic enzyme in 3 rats; 5 non-injected rats. After 5-7 weeks, rats were injected with apomorphine (0.4 mg/kg, subcutaneously) and rotational asymmetry determined in each group just prior to sacrifice. After the brains were fixed and sectioned, the SN, and forebrain were stained immunohistochemically for tyrosine hydroxylase immunoreactivity (THir). See Section 3.

Determination of rotational asymmetry. Rats first were injected subcutaneously with the dopamine agonist apomorphine (0.4 mg/kg) dissolved in 0.1 ascorbate saline solution. After waiting 5 min in their home cage, they were placed in a hemispheric rotation bowl 40 cm wide and 20 cm deep. Rotational behavior was then determined for 30 min and the number of ipsilateral and contralateral turns quantified by observation.

### Example 1

### DOPAL is a Major DA Metabolite in Human Brain

Levels of 3,4-dihydroxyphenylacetaldehyde (DOPAL), dopamine (DA), homovanillic acid (HVA) were determined in substantia nigra (SN), caudate and putamen from a 67-year-old man with a post-mortem interval of 8.3 h using MHPLC-EC (Table 1). Values are means±SE of at least six replicates of each chemical. Results indicate that DOPAL is a major dopamine metabolite in human SN and its projections.

TABLE 1
Levels of DOPAL, DA and-HVA in human
substantia nigra and striatum.

| | Brain tissue (pg/mg wet weight) | | |
|---|---|---|---|
| Compound | Caudate | Putamen | Substantia Nigra |
| DOPAL | 149 ± 3.7 | 120 ± 2.9 | 397 ± 4.8 |
| HVA | 113 ± 2.7 | 132 ± 3.5 | 321 ± 4.9 |
| DA | 191 ± 4.4 | 99 ± 2.5 | 275 ± 4.9 |

### Example 2

### DOPAL is Toxic in Vitro PC-12 Cells

DOPAL, as well as other physiologically metabolites and adducts, were added to PC12 cells in culture at concentrations that were physiologically relevant. The results indicated that DOPAL, but not Dopamine, Homovanillic acid, DOPAC or Tetrahydropapaveroline were toxic in vitro at physiological levels (Table 2).

TABLE 2
DOPAL, Dopamine, Homovanillic acid,
DOPAC and Tetrahydropapaveroline were
added to PC12 cells in culture.

| | |
|---|---|
| Compound added | PC 12 cells/well × 103 |

Experiment 1

| | |
|---|---|
| None | 299 ± 19 |
| DOPAL (66 μM) | 100 ± 5a |
| Dopamine (66 μM) | 329 ± 21 |
| Homovanillic acid (66 μM) | 322 ± 20 |
| DOPAC (66 μM) | 334 ± 10 |
| Tetrahydropapaveroline (66 μM) | 346 ± 15 |

Experiment 2

| | |
|---|---|
| None | 143 ± 12 |
| DOPAL (30 μM) | 107 ± 10b |
| DOPAL (30 μM + rotenone 10 μM) | 61 ± 9c |
| Rotenone (10 μM) | 127 ± 9 |

Experiment 3

| | |
|---|---|
| None | 141 ± 22 |
| DOPAL (6.6 μM) | 105 ± 12b |

### Example 3

### DOPAL Triggers Aggregation of Purified α-Synuclein in Test Tube Experiments

Western blot analysis was performed to analyze DOPAL-induced aggregation of α-synuclein (FIG. 1). DOPAL was dissolved in 1% benzyl alcohol, then diluted to a final concentration of 1.5-, 500 μM. AS (2 μM) was incubated at 37° C. in 20 μl of 100 mM tris-HCl buffer (pH 7.2) with or without DA, DOPAL (1.5-1,500 μM), DOPAC, or homovanillic acid (HVA) for up to 4 h. The reaction was stopped by heating at 70° C. for 3 min in SDS buffer. Results indicated that DOPAL, but not DA or its other metabolites, triggers dose-dependent aggregation of α-synuclein oligomers of increasing size in vitro. (see Burke et al. Acta Neuropath, 115:193-203, 2008)

### Example 4

### DOPAL Triggers α-Synuclein Aggregation in Vivo

The SN of rats were injected with either 1 μg DOPAL or vehicle control to determine the effects of DOPAL on α-synuclein aggregation in vivo (FIG. 2). The rats were sacrificed and SN biopsied. After PAGE and immunoblotting with AS 202 antibody, the blot was striped and re-probed with antibody against β-actin. Results after 1 and 4 hours were compared. Results show that DOPAL triggers a selective dose and time dependent aggregation of α-synuclein but not of β-actin monomer to oligomers in vivo (FIGS. 2A and 2B). (see Burke et a1. Acta Neuropath 115:193-203, 2008.

### Example 5

Neuropathological Evaluation: Immunohistochemistry In all cases there was a decrease in immunoreactivity of TH in the SN ipsilateral to the injections of DOPAL (FIG. 3B, yellow arrowhead) compared to the contralateral, non-injected side (FIG. 3A). There also was significantly (p, 0.001) less TH immunoreactivity in the striatum on the side ipsilateral to the DOPAL injections (FIG. 3D, arrows; FIG. 3E) compared to the noninjected contralateral side (FIG. 3C, arrows; FIG. 3E). After background densities were subtracted, the inventors calculated a 28% reduction in immunoreactivity in the striatum on the side ipsilateral to the DOPAL injections, suggesting a loss of DA terminals on the injected side. It was noted that the ventrolateral striatum through levels of the globus pallidus were especially denervated (FIG. 3D, red circles). Spot density measurements contralateral (17.864.5 units) versus ipsilateral to the DOPAL injections (3.565.9 units) here were reduced 80%.

Neuropathological Evaluation: Stereology The SN was included in 8-10 sections of all cases counted, and its total length was approximately 1.25 mm. Mean volume of the SNpc of control rats was 268,639,250 m³, while that of the SNpr was 777,696,500 m³. Mean volume of the SNpc in the DOPAL injected rats was 264,674,833 m³ while that of the SNpr was 760,212,500 m³. There was no significant difference in mean volumes of SNpc or SNpr between controls and DOPAL injected rats. The inventors first counted TH immunoreactive neurons in the SNpc on the side of the DOPAL injection and compared them to those on the non-injected side. When only TH immunoreactive neurons were counted, the mean number of TH immunoreactive neurons ipsilateral to the DOPAL injections side was 50% less than that of the contralateral non-injected side, significantly different (p=0.032) using the paired samples T-test by difference method (Table 3). However, the inventors noted that numerous SNpc neurons sometimes were not stained for TH despite robust labeling of others (FIG. 3F). Thus, the inventors compared the number of Nissl stained profiles in sections immunostained with a-syn rather than TH in the SNpc's ipsilateral to the DOPAL injections to those of control rats which had received injections of a buffered saline solution into their SN's (Table 3). The number of Nissl-stained neurons in the SNpc (compare FIGS. 4A, B) of the DOPAL injected rats was 43% less than that of the saline-injected rats (FIG. 4C) which was significantly different (p=0.001). The inventors then determined whether DOPAL was toxic to neurons in the subjacent pars reticulata of the SN. The number of neurons in the SNpr of the DOPAL-injected rats was not different from the saline-injected rats (Table 3; FIG. 4C). This suggests that DOPAL is selectively lethal to dopaminergic neurons in the SNpc.

TABLE 3
Toxic Effect of DOPAL on Substantia Nigra Neurons.

| | TOTAL SNpc | TOTAL SNr | TH SNpc |
|---|---|---|---|
| | Neurons | Neurons | Neurons |
| Control | 11926 (1084) | 12422 (832) | 11969 (3699) |
| Experimental | 6879 (422) | 11761 (715) | 5884 (1365) |
| p-value | 0.001 | n.s. | 0.032 |

The effect of DOPAL injections into the substantia nigra on neurons in either the pars compacta (SNpc) or the pars reticularis (SNpr) are shown. Control animals were injected with buffered saline while experimental animals were injected with DOPAL (4 mg/800 nl). Unbiased stereology was used to assess the number of neurons (see Methods). Means (SD); n.s.=not significant.

### Example 6

### Injections of Either DOPAL or ALDH Antisense Oligonucleotide into Rat Substantia Nigra Produces a Behavioral Model of Parkinson's Disease

Rotational asymmetry was assessed to quantify the effect of unilateral depletions of striatal dopamine from disruptions of nigrostriatal circuitry. The rotational asymmetry behavior of rats (n=6) changed dramatically after injections with DOPAL (FIG. 5). Tests in control rats (n=4) showed little right-left preference for turning. However the DOPAL injected rats excited by apomorphine (0.4 mg/kg) just prior to their sacrifice significantly preferred turning towards the side of the injection. Rats significantly (p, 0.05) prefer rotating to the side ipsilateral to the unilateral DOPAL injections versus control rats (FIG. 5) after injections of apomorphine.

### Example 7

ALDH1A1 antisense oligonucleotide injections into rat substantia nigra trigger loss of DA SN neurons and their basal forebrain projections and produce a behavioral model of PD. An antisense compound was used to decrease expression of ALDH1A1, which is responsible for breaking down DOPAL within the neuronal cell body and its effects on dopamine SN neurons and behavior were examined (FIGS. 6, 7). Three unilateral injections (total 800 nl; 700 pg/nl) of an antisense compound oligonucleotide (SEQ ID NO: 1), directed against the enzyme ALDH1A1, and comprising a phosphorothioate backbone, were made into the substantia nigra nucleus of the rat. The injections were placed in the pars compacta portion of the substantia nigra, the subnucleus where most of the dopaminergic neurons are found. While the intensity of staining of the TH was slightly less in the injected SN (arrow; right side), dopaminergic projections to more rostral parts of the brain were dramatically reduced. Note the loss of immunoreactivity in the ventral pallidum and olfactory tubercle in C (arrows) and the shell of the nucleus accumbens and olfactory tubercle in D (arrows) after these injections. The immunoreactivity in the striatum of the injected side also was less dense in both cases (not shown). An adjacent section immunostained with antibodies showing neuronal cell bodies (Neun) is shown from case R2504. Note neurons immediately adjacent to the injection site (marked with red beads) have died, but there are numerous neurons in the subjacent pars reticulata of the SN (arrow).

The rat also showed dramatic shifts in its rotational preference after injections either antisense 1 (SEQ ID NO: 1) or antisense 2 (SEQ ID NO: 3) (FIG. 7). Behavioral asymmetry is considered the standard behavioral test for rodent models of Parkinson's disease. The column on the right is a summary of all the behaviors recorded in the rotational asymmetry test for all the rats (n=7). It was noted that control data obtained prior to antisense injections (n=7) showed little bias towards turning either to the right or left, but antisense oligonucleotide treated rats excited with apomorphine (0.4 mg/kg) just prior to sacrifice dramatically preferred turning towards the side of the antisense injection. Results show that loss of DA SN neurons and their basal forebrain projections after ALDH1A1 antisense oligonucleotide injection is accompanied by behavioral changes used in standard animal models of PD.

### Example 8

Antisense inhibition of ALDH1A1 expression in rats. Rats were injected on the ipsilateral side with two different antisense, Antisense 1 (SEQ ID NO:1and Antisense 2 (SEQ ID NO:3) which are complementary to ALDH1A1 mRNA. Tissue was harvested from the ipsilateral and contralateral sides after 1 and two weeks. The tissue was homogenized and 1 μg of protein was subjected to immunoblotting with antibody directed against ALDH1A1 (FIG. 8). Loss of ALDH1A1 in the ipsilateral sides treated with either Antisense 1 or Antisense 2 compared to the contralateral control at either 1 week or 2 weeks was apparent.

The immunoreactive bands of the Western blot of FIG. 8 were analyzed by Unscan it soft ware (Silk Scientific) and their optical densities plotted in FIG. 9. The results quantitate and confirm the apparent loss of ALDH1A1 on the ipsilateral sides treated with either Antisense 1 or Antisense 2 compared to the contralateral control at either 1 week or 2 weeks.

All publications and patents cited in this specification are hereby incorporated by reference in their entirety. The discussion of the references herein is intended merely to summarize the assertions made by the authors and no admission is made that any reference constitutes prior art. Applicants reserve the right to challenge the accuracy and pertinence of the cited references.

What is claimed is:

1. An antisense compound consisting of at least 8 contiguous nucleic acid residues complementary to ALDH1A1 mRNA, and conservatively modified variants thereof, whereby the antisense compound reduces levels of ALDH1A1 protein when administered to a mammalian cell.

2. The antisense compound of claim 1, consisting of at least 10 contiguous nucleic acid residues complementary to ALDH1A1 mRNA,

3. The antisense compound of claim 1, consisting of at least 14 contiguous nucleic acid residues complementary to ALDH1A1 mRNA,

4. The antisense compound of claim 1, consisting of at least 18 contiguous nucleic acid residues complementary to ALDH1A1 mRNA.

5. The antisense compound of claim 1, consisting of at least 21 contiguous nucleic acid residues complementary to ALDH1A1 mRNA.

6. The antisense compound of claim 1, wherein then the ALDH1A1 mRNA consists of coding ALDH1A1 mRNA.

7. The antisense compound of claim 1, wherein then the ALDH1A1 mRNA consisting of residues 313 to 333 of SEQ ID NO: 2.

8. The antisense compound of claim 1, wherein the ALDH1A1 mRNA consists of a non-coding ALDH1A1 mRNA.

9. The antisense compound of claim 1, wherein the ALDH1A1 mRNA consists of the protein start site.

10. The antisense compound of claim 1, wherein the ALDH1A1 mRNA consisting of residues 22 to 43 of SEQ ID NO: 2.

11. The antisense compound of claim 1, wherein the antisense compound is selected from the group consisting of SEQ ID NO: 1 and SEQ ID NO: 3.

12. An animal model for Parkinson's disease comprising, a non human animal, and an effective amount of antisense compound as set forth in claim 1, whereby the effective amount of antisense compound is administered to the non-human animal and the non-human animal exhibits symptoms of Parkinson's disease.

13. The animal model of claim 12 whereby the non-human animal is a rat and the antisense compound is administered by injection into the substantia nigra of the brain.

14. An animal model for Parkinson's disease whereby a non-human animal is genetically engineered to express reduced amounts of ALDH1A1 through deletion or inhibition of ALDH1A1 mRNA, and the non-human animal exhibits symptoms of Parkinson's disease.

15. A method of testing a potential therapeutic agent for Parkinson's disease comprising,
a) administering the potential therapeutic agent to an animal exhibiting a Parkinson's disease state, and
b) assessing the behavioral, biochemical or histological changes in the animal compared to animals exhibiting a Parkinson's disease state but not administered with the potential therapeutic agent.

16. The method of testing a potential therapeutic agent of claim 15 whereby assessing the behavior change consists of assessing Rotational behavior.

17. The method of testing a potential therapeutic agent of claim 15 whereby assessing the histological change consists of measuring loss of substantia nigra dopamine neurons.

18. The method of testing a potential therapeutic agent of claim 15 whereby assessing the biochemical changes consists of assessing α-synuclein aggregation.


SEQUENCE LISTING (3 sequences)

SEQ ID NO: 1 - DNA, 22 bp, Rattus norvegicus
tgcaggggaagacattgctggt

SEQ ID NO: 2 - DNA, 2095 bp, Rattus norvegicus
  ref <308>: DATABASE ACCESSION NUMBER: NCBI/NM_022407.3,
  ref <309>: DATABASE ENTRY DATE: 2010-05-13
  ref <313>: RELEVANT RESIDUES IN SEQ ID NO: (1)..(2095)
cacctgtgttccaggagccaaaccagcaatgtcttcccctgcacagcctgcagttcctgccccactggccaacttgaagattcaacacaccaagatcttcataaacaatgaatggcatgattcagtgagtggcaagaaatttcctgtccttaaccctgcaactgaggaggtcatctgccatgtggaagaaggggacaaggcagatgttgacaaagctgtgaaggccgcaagacaggctttccagattggctccccatggcgcaccatggatgcttcagagagaggacgcttgctgaacaagctggctgacttaatggagagagatcgtctgctgctggctacaatagaggccatcaatggtgggaaagtctttgccaatgcgtacttgtcggatttaggaggcagcataaaagcattaaagtactgtgcaggctgggctgacaagatccatggtcaaacaataccgagtgatggagacattttcacttttacgagacgtgaacctattggggtgtgtggccaaatcatcccttggaattttccactgcttatgttcatttggaagataggccctgccctcagctgtgggaacacggtggtcgtcaagccagcagagcaaactcctctcactgctcttcacatggcatctttaataaaagaggcagggtttcctcctggcgtggtgaacattgtccctggttatgggccaactgcgggggcagcgatctcctctcacatggatgtggacaaggtggccttcacgggatcaacccaggttggcaaattaatcaaggaagctgcaggaaaaagcaatctgaagagggtcacgctggagcttgggggaaagagcccttgcattgtgtttgcagatgccgacttggacattgctgtcgagtttgcacaccatggagtcttctaccatcaaggccaatgctgcgtcgcggcatcccggattttcgttgaggagtcagtttatgatgagtttgtgagaaagagtgttgagcgagccaagaaatatgtccttggaaaccctctgacccaaggaataaatcagggccctcagattgacaaggagcaacatgataaaatccttgatctcattgagagtgggaagaaagaaggagccaaactggagtgtggtggaggacgctgggggaacaaaggcttctttgtccagcccacagtcttctccaatgtgaccgatgagatgcgcattgccaaagaggagatatttggaccagtgcaacaaatcatgaagtttaagtccatagatgatgtgatcaagagagcaaacaatactacctatggtctagcagcaggagtcttcacaaaagacctggacagggccatcactgtgtcttctgctctgcaggccggggtagtgtgggttaactgctatatgatcttgtcagcccagtgccccttcggtggattcaagatgtctggaaatggacgagaactgggtgaacatggtctttatgagtacactgagctcaagacagttgcaatgaaaatatctcagaagaactcctaagaagcagagtgaagagaaactctcagctgtggctacacgtctcctatcgtcaccagcaaagtgttgttttactataattttttcttctgttgatttctttaacatgatgaatccatcagtgttactgttactcatagaaaacatgtagcttaatcctacaaaaccactcaccttctaatatgtgactccagtccttatcccagaataaaaggatagatttaggtgcaagctctctgtaactctgtcatgataggtgctttctgtcgtagctccctgtctagagtactcatttggtgaggaggaccagtcgtgatttaagctctgtccctctgtgaccccttgaactgcttctcggcatgcatgataactgcagagtcggctgctctgtttcccaggtgttgtgaaatgttttctagaaagccatgcctgcttatcaaatgaaatgcccagctgtaattagaatgcaaagctaataaagggacccttgcatgattttgttggtctgtaattatttgggaatcaactaggattatggcaataaactctgctggtcaaaaaaaa

SEQ ID NO: 3 - DNA, 21 bp, Rattus norvegicus
agcagacgatctctctccatt